(19) United States
(12) Patent Application Publication
(10) Pub. No.: US 2012/0232630 A1
(43) Pub. Date: Sep. 13, 2012

(54) ARTICULATING INTERFACES FOR BIOLOGICAL TISSUES

(76) Inventor: Eugene Dariush Daneshvar, Ann Arbor, MI (US)

(21) Appl. No.: 13/414,134

(22) Filed: Mar. 7, 2012

Related U.S. Application Data
(60) Provisional application No. 61/449,913, filed on Mar. 7, 2011.

(51) Int. Cl. A61N 1/05; A61N 1/04

(52) U.S. Cl. 607/116; 607/149

(57) ABSTRACT

A biological tissue interface system is disclosed which employs an electroactive polymer actuator to pivot a tissue interface portion which may be adapted to engage with neural tissue. The system comprises a base portion, an articulating portion having a proximal end attached to the base portion and a free distal end, and an actuator operably coupled to the articulating portion, driven by a conjugated polymer that changes dimension in response to an electric charge. The polymer applies a force to pivot the articulating portion, relative to the base portion, to engage tissue at the distal tissue interface. In neural interface applications, the articulating portion may comprise at least one conducting surface for neural communications, such as a microelectrode or a polymer.

### CROSS REFERENCE TO RELATED APPLICATIONS

This application claims priority to Provisional Patent Application No. 61/449,913 filed Mar. 7, 2011, the entire disclosure of which is hereby incorporated by reference and relied upon.

### FEDERALLY SPONSORED RESEARCH OR DEVELOPMENT

Pursuant to 35 U.S.C. §202(c), the United States government may have an interest in the invention described herein, which was made in part with funding from the Center for Neural Communication Technology, a P41 Resource Center funded by the National Institute of Biomedical Imaging and Bioengineering (NIBIB, P41 EB002030), and supported by the National Institutes of Health (NIH). This work was also supported by the Department of Defense Multidisciplinary University Research Initiative (MURI) program administered by the Army Research Office under grant W911NF0610218.

### BACKGROUND OF THE INVENTION

1. Field of the Invention

The present invention relates to medical devices, and more particularly to micro-electrical-mechanical systems (MEMS) using electric charge and conjugated polymers to engage biological tissue.

2. Related Art

Use of conjugated polymers for use in biomedical applications is a relatively new but rapidly developing area. Certain conjugated polymers undergo dimensional changes when ions transfer into or out of the polymer. The unique properties of conjugated polymers have prompted research into using them as actuators for use as micro-muscles, tiny steerable micro-catheters, endoscopes that can be guided inside blood vessels or along the spinal cord, and micro-fluidic devices.

Electroactive polymers, also referred to as “conjugated polymers” or “conductive polymers” or “conducting polymers,” are characterized by their ability to change shape in response to electrical stimulation, such as applying a voltage potential or current or electric field at the polymer. Conducting polymers typically have a conjugated backbone and increase electrical conductivity under oxidation or reduction. Some common conjugated polymers are polyaniline, polypyrrole (PPy) and polyacetylene. These materials are typically semi-conductors in their pure form. Oxidation or reduction promotes higher conductivity, an electron charge imbalance, and ion flow into or out of the material in order to balance charge. Ions, or dopants, may enter the polymer if ions are present in the immediate vicinity of the polymer, such as a conductive electrolyte medium. On the other hand, if ions are already present in the polymer when it is oxidized or reduced, they may exit the polymer in the presence of voltage. In some conjugated polymers, expansion (or contraction) is due to ion insertion (or deletion) between chains. The dimensional change may be more pronounced where the transferred ion is bound with larger molecules. In other polymers interchain repulsion may be the dominant effect.

The conjugated polymer may be coupled with a substrate material to form a controllable actuator. The observed magnitude of the dimensional change of a conjugated polymer and actuation force will vary depending on the composition of the conjugated polymer and the substrate on which it is deposited, their dimensions and thickness, polymer deposition and manufacturing/preparation techniques, the ion environment adjacent to the polymer, the voltage applied, and the number of actuation cycles. Conjugated polymers are a potentially good candidate for micro-scale actuation of biomedical devices because they may be integrated and fabricated with an underlying substrate and larger MEMS system, actuation typically requires low voltage (+/−1 V), and they can be made from biologically stable materials. Moreover, a MEMS biomedical system employing a conjugated polymer can be controllably actuated on a micro (less than 2 mm) scale.

Neural interface devices pose problems that may benefit from a conjugated polymer actuation system. Microelectrodes placed in the vicinity of nerves may function as nerve stimulators, sensors, or both. A challenge in this area is in creating and maintaining a healthy biotic interface between the targeted nerves and the device. The body tends to react to an implanted device by treating it as a foreign object, and will typically initiate an immune response and surround the device with encapsulating cells. The body's immune response may cascade, causing chronic encapsulation and irreversible neural cell damage at the biotic interface. Such reactions tend to degrade the signal to noise ratio (SNR) of signals received by or transmitted from the neural device. SNR also degrades as distance increases between the electrode interface and the target neural site. Where the device is configured to transmit signals to neural tissue, increasing the voltage of the signals to reach the targeted nerves can lead to other deleterious effects, including unwanted stimulation of surrounding tissue. Where the device is functioning as a receiver of neural signals, close proximity and good SNR are especially desirable. Further, it is observed that larger devices cause more biological trauma along the insertion path and at the target site, exacerbating the body's immune response.

Securing a neural interface to the target area presents further challenges. Even if initially well-placed, if the interface is insecurely attached to the biological tissue it may move or dislodge entirely, resulting in faulty signal communication and degraded SNR. Attachment requirements may differ depending on the area of the body in which the interface operates. Securing an interface within an organ, e.g., a deep-brain implant, will require different attachment techniques than securing a device to the surface of an organ, e.g., electrocorticography (ECOG) electrodes on the brain, or retinal implants on the retina. Attachment requirements to a peripheral nerve, e.g., along an arm, may differ from organ attachment techniques, or attachment along the spinal cord. Common to all such applications, however, is the desirability of securing the interface close to the targeted neural tissue.

US 20100016957 (Jager et al.) involves using electroactive conjugated polymers in biomedical devices. Jager discloses embodiments which control microdelivery of drugs and act as micro surgical tools, such as micro-forceps. Jager also discloses using electroactive polymers to deliver micro quantities of drugs to neural tissue, and to bind or splice torn or cut neural tissue. However, Jager does not disclose using conductive polymers to place microelectrodes in communication with neural tissue, or implantable, intra-tissue articulating portions that penetrate biological tissue.

US 20100268055 (Jung, et al.) discloses a MEMS neural electrode that may be articulated from a substrate. However, Jung does not disclose use of electroactive polymers to actuate the articulated portion, but rather at least two layered materials having different thermal expansion coefficients; Jung's device is pivotally actuated, for example, by changing the ambient temperature around the device.

US 20100268312 (Wallace et al.) discloses an electroactive polymer actuator coupled with a plurality of electrodes engageable with neural tissue in the cochlea portion of the ear. However, Wallace's system does not disclose articulating portions that pivot from a base portion, and in any case is many times larger than the micro-scale neural interface system disclosed by the present invention.

It would be useful to have a MEMS system having minimal size yet controllable tissue engagement, enabling a precise interface with targeted biological tissue such as neural tissue. It would be useful if the system comprised multiple actuators that could be collectively or independently moved, with multiple independently operable interface areas. Such devices would, for example, facilitate placement of electrodes proximate to the intended neural tissue, and further permit selective stimulation of proximal neural tissue. Further, such a system could be configured to grasp biological tissue and secure the device at the target area, further improving SNR and reducing or eliminating the need for sutures, adhesives, or other attachment techniques.

### SUMMARY OF THE INVENTION

A biological tissue interface system is disclosed which employs an electroactive polymer actuator to pivot a tissue interface portion. Such a system may be particularly well adapted to interface with neural tissue. In one embodiment, the system comprises a base portion having a base substrate, an articulating portion having a proximal end attached to the base portion and a free distal end, and an actuator operably coupled to the articulating portion, driven by a conjugated polymer that changes dimension in response to an applied voltage potential, an electric current, electric field, or electric charge. The polymer applies a force to pivot the articulating portion, relative to the base portion, from a first position to a second position. The articulating portion has a tissue interface located at its distal end. In neural interface applications, the distal end may have at least one microelectrode or polymer conductor engageable with neural tissue.

### BRIEF DESCRIPTION OF THE DRAWINGS

These and other features and advantages of the present invention will become more readily appreciated when considered in connection with the following detailed description and appended drawings, wherein:

FIG. 1 is a top view of the base portion substrate and articulating portion substrate of an articulating biological tissue interface.

FIG. 2 is a top view of the base and articulating portions showing an electrical contact layer for the actuator, two electrical traces on the substrates, and a neural microelectrode.

FIG. 3 is a top view of the base and articulating portions showing a conjugated polymer layer and insulation of the electrical traces.

FIG. 4A is a sectional view of FIG. 3 showing the articulating portion pivoted away from the base portion in a substantially non-planar position.

FIG. 4B is different sectional view of FIG. 3.

FIG. 5 is a top view of an embodiment of the invention in the form of a grid array of articulating biological interfaces.

FIG. 6 is a sectional view of the grid array in FIG. 5 showing interfaces pivoted away from the base portion.

FIGS. 7A and 7B are side views of another embodiment showing a system partially and fully inserted into biological tissue with a plurality of articulating portions grasping biological tissue and functioning to communicate neural signals.

FIGS. 8A and 8B are side views showing another embodiment of a grasping system showing pre-attachment and post-attachment positions of articulating portions.

FIG. 9A is a front view of another embodiment of a system adapted to grasp biological tissue with intra-tissue articulating portions, and optionally communicate neural signals.

FIG. 9B is a perspective view of FIG. 9A.

FIGS. 10A, 10B and 10C are perspective views of another embodiment of a system showing a compound articulating portion, having an articulating tip segment and an articulating shank segment.

FIG. 11 is a top view of another embodiment showing a grid array of neural interfaces combined with peripheral articulating interfaces adapted for grasping biological tissue.

FIG. 12 is a sectional view of the grid array of FIG. 11, prior to attachment to proximate biological tissue and showing grasping interfaces pivoted away from the base portion.

FIG. 13 is a perspective view of another embodiment showing a grid array configured as a cuff electrode, with a plurality of actuators coupled to the base portion and a plurality of neural electrode sites.

FIG. 14 is a schematic of the invention illustrating a retinal application.

FIG. 15 is an enlarged perspective of FIG. 14 showing a grid array of neural electrodes adjacent the rear retinal wall.

FIG. 16 is a side view of another embodiment of a system having two pairs of microelectrodes arranged on two articulating portions, with each microelectrode pair configured to generate a voltage potential or pass electric current between them.

FIG. 16A is a second view of FIG. 16 with a both articulating portions pivoted away from the base portion, thereby steering the electric current/voltage into a different position.

FIG. 17 is another embodiment of a system similar to FIGS. 16 and 16A, showing current/voltage passing between the two articulating portions.

FIG. 18 is another embodiment showing a stacked system of microelectrodes on the base and articulating portions.

FIG. 18A is a second view of FIG. 18 with the articulating portions pivoted to steer or apply electric current/voltage distant from the base portion.

FIG. 19 is another embodiment of a system similar to FIGS. 18 and 18A showing other current/voltage configurations included within the scope of the invention.

### DETAILED DESCRIPTION OF PREFERRED EMBODIMENTS

After first briefly describing conjugated polymers and the challenges of interfacing with neural tissue, this description will focus on an exemplar embodiment for providing a microelectrode site with a projection that can be actuated via a conjugated polymer from the surface of a base substrate to reduce the distance to a neural interface target. This feature may improve the neural interface by optimizing the electrode-tissue distance or by distancing the electrode site from the base substrate when favored. Subsequently, other examples of MEMS devices utilizing polymer actuators to engage with neural and other biological tissue will be described.

Electroactive polymers, also referred to as “conjugated polymers” or “conductive polymers” or “conducting polymers,” are characterized by their ability to change shape in response to electrical stimulation. They typically have a conjugated backbone and the ability to increase electrical conductivity under oxidation or reduction. Some common electroactive polymers are polypyrrole (PPy), polyaniline, and polyacetylene. These materials are typically semi-conductors in their pure form. However, upon oxidation or reduction of the polymer, conductivity is increased. The oxidation or reduction leads to a charge imbalance that, in turn, results in a flow of ions into or out of the material in order to balance charge. These ions, or dopants, enter the polymer from an ionically conductive electrolyte medium that is coupled to the polymer surface. If ions are already present in the polymer when it is oxidized or reduced, they may exit the polymer.

Conjugated polymer performance is highly dependent on its synthesis and the medium in which it is employed. Each new blend or application needs to be characterized to ensure that it performs as expected. PPy is substantially biocompatible, requires low power to undergo dimensional change, and has other features that enable fabrication according to the current invention. PPy is characterized by alternating single and double bonds along the polymer backbone. A large counter anion such as dodecylbenzenesulfonate (DBS) may be used to polymerize PPy. The DBS becomes embedded into the polymer matrix leaving only the influx and efflux of hydrated cations in the solution as the main mechanism of actuation.

Where the conjugated polymer is present in an environment having ions with hydration shells, such as human body tissue, a mass movement of ions and their hydration shells moving in and out of the polymer matrix causes change in polymer volume. Oxidation, or removal of electrons, can be electrochemically achieved by the application of a sufficiently positive voltage potential or electric charge (e.g. current and/or electric field). To maintain charge neutrality after oxidation, the polymer seeks to incorporate negatively charged ions to compensate for these positive charges on the polymer backbone. Smaller cations, being more electronegative and surrounded by larger hydration shells, produce greater force and displacement in the polymer during reduction. Smaller anions, such as Cl—, are also known to participate in charge balancing in the PPy, but don't have as large of an effect on volume change. Divalent cations may enter the polymer when present but are known to be less mobile because of their stronger binding forces.

If a negative voltage potential is applied to the polymer in the presence of ions, reduction of the polymer occurs, and the polymer seeks to shed negatively charged ions. Thus the conjugated polymer component in the presence of electrical or electronic stimulation can effectuate an actuation, effectively a reversible volume change in the polymer matrix and/or ion flux with the surrounding electrolyte medium. This behavior of the conjugated polymer can be exploited to bend a substrate on which the polymer is placed. In addition to PPy, any conjugated polymer that exhibits contractile or expanding properties may be used within the scope of the invention. Polyaniline is another example of such a usable conjugated polymer.

From an energy and safety standpoint, it may be preferable to configure the devices of the present invention such that the electroactive polymers expand where no potential is applied (i.e., under steady state conditions) and constrict upon the application of an appropriate voltage, or vice versa depending on the application. The polymer composition and its preparation can often be modified to achieve the desired steady-state configuration. Given the small size of the actuators, the embodiments presented herein contemplate using the body environment as the electrolyte source, with ionic species and their species concentrations present in the biological tissue environment.

The disclosed invention may be particularly useful to interface electric signaling systems (adapted either to emit or receive signals) with neural tissue. An effective interface for neural stimulation and recording are strongly dependent on the physical proximity of the electrode to the neurons targeted. Both recording quality and stimulation therapy are subject to the proximity of the electrode to the intended target, where attenuation is caused by the diffusivity of the signal as a function of distance in the application medium. Signal attenuation within the brain has been shown to be inversely proportional to the distance squared. Unfortunately the inflammatory response to the devices also frequently disrupts normal neuronal density. Likewise, in the eye, gaps that occur between the retina and stimulating electrode array are detrimental to efficacy of the therapy. Similarly, within cochlear implants the separation distance between the electrode array and the neural receptors along the cochlear wall increases the stimulus levels required which limits the electrode density and consequently decreases the frequency resolution of hearing.

In order to mitigate the immune response, anti-inflammatory coatings such as dexamethasone and substrates made from parylene and polyethylene glycol have been used to make a variety of devices more biocompatible. Conjugated polymers and carbon coatings have been used to reduce electrode site impedances while increasing charge transfer for stimulation. Also, attempts of seeding growth factors and stem cells on the probe to promote new neuron growth have also been studied as methods to bridge the electrode interface gap. Nevertheless, mitigating the underlying immune response remains a challenge.

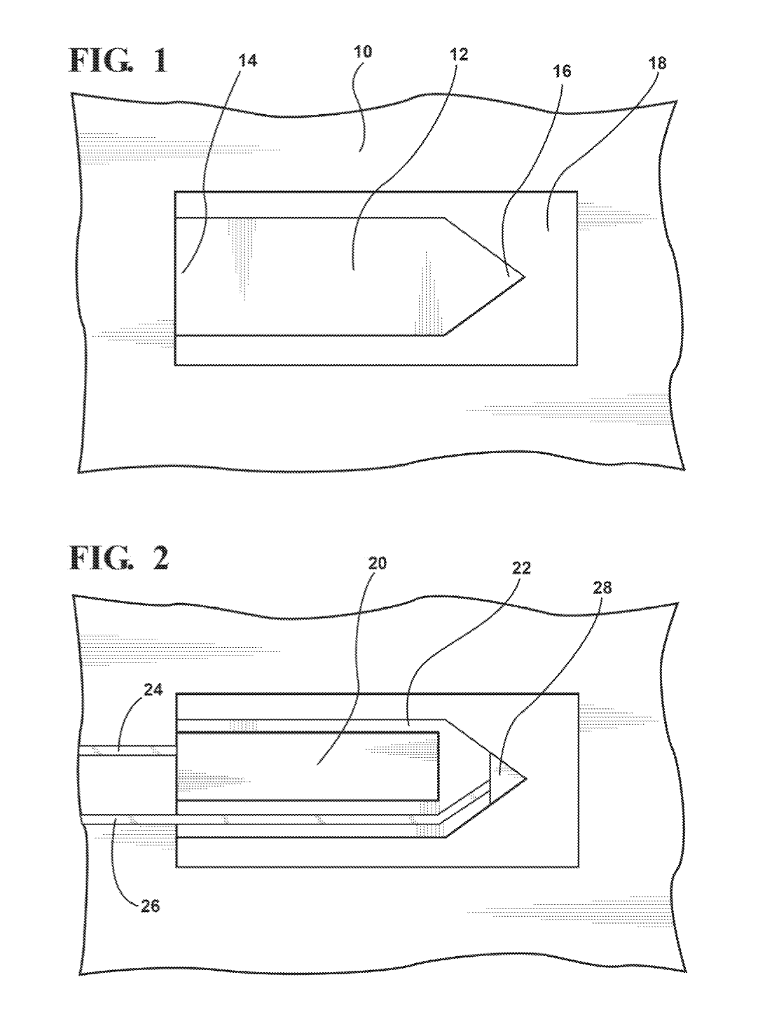

Turning to the drawings, FIG. 1 shows a top view of a portion of a biological tissue interface system comprising a base portion 10 and articulating portion 12 and an opening 18 preferably surrounding articulating portion 12 on three sides in plane. Articulating portion 12 has a proximal end 14 and a distal end 16. For purposes of illustration, distal end 16 is shown with a pointed tip. However, the distal end of the articulating portion could be square, oblong, semi-circular, or any other configuration. Further, distal end 16 could be configured as a barb, analogous to an anchor or fishhook, to better secure the interface portion of articulating portion 12 when it engages with biological tissue.

Turning to FIG. 2, the same top view of the same portion of the biological tissue interface system is shown with additional structural detail. Articulating portion 12 is comprised of an articulating substrate 22. In microfabrication, an electrical contact layer 20 is deposited on top of articulating substrate 22. FIG. 2 also shows a first electrical trace 24 and a second electrical trace 26. The first electrical trace 24 is connected to the electrical contact layer 20. The second electrical trace 26 is electrically isolated from electrical trace 24 and the electrical contact layer 20. The second electrical trace 26 is connected to a microelectrode 28 at the distal end 16 of articulating portion 12. As will be discussed below, certain aspects of this invention do not require a microelectrode 28 at the tip of the articulating portion 12, and likewise do not require a second electrical trace. Where a neural interface is desired, however, a conducting surface, such as a microelectrode 28 coupled with an electrical trace 26 may be fabricated on the articulating portion 12. In another embodiment more fully discussed below, conjugated polymer 30 may itself serve as a conductive surface for the neural interface, as well as an actuator. Those familiar with the state of the art recognize that other neural interfaces may be substituted for electrical traces and microelectrode sites, such as optical waveguides used in optogenetics. Similarly, microelectrodes may be conditioned to detect chemical signals instead of electrical signals. In this application, “conductive surface” includes transductive means of communicating neural signals via optical waveguides or chemical detection.

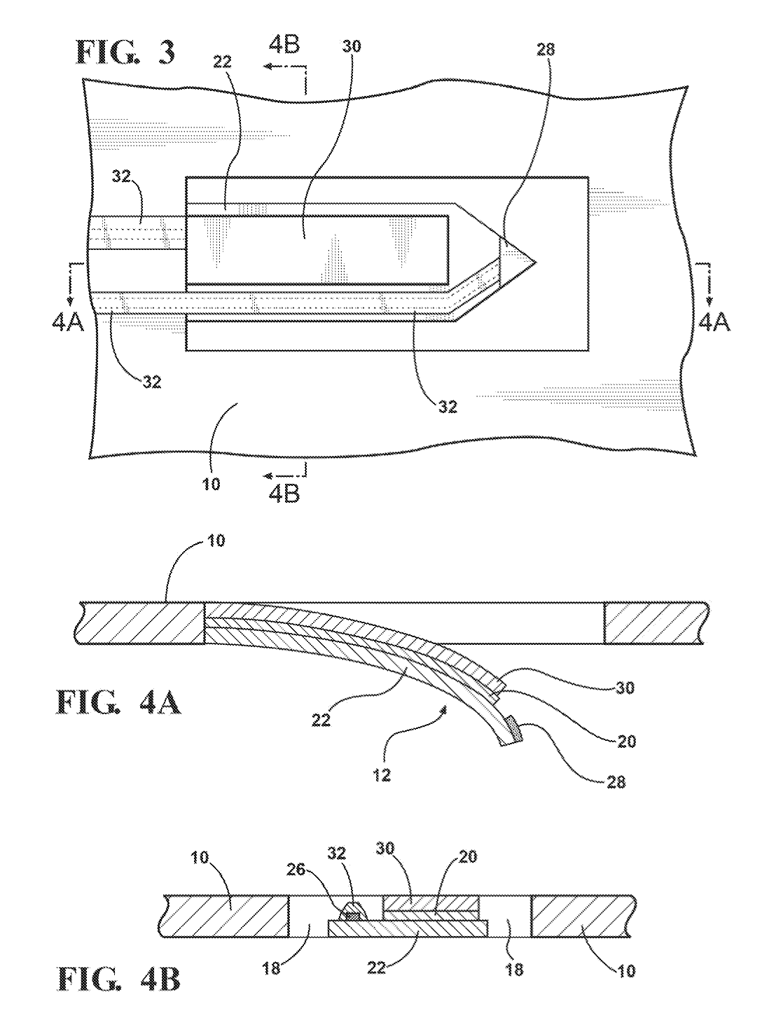

FIG. 3 is another top view of a portion of the biological tissue interface system showing a conjugated polymer 30 deposited on top of electrical contact layer 20 (not shown). It also shows trace insulation 32 deposited over electrical traces 24 and 26 to electrically isolate the actuation electrical system from the neural signal system. It will be appreciated that instead of a separate layer of trace insulation 32, the electrical traces may in some cases be insulated by embedding them into the base portion substrate and/or articulating portion substrate 22.

FIG. 4A is a sectional view of the biological tissue interface system shown in FIG. 3. Articulating portion 12 is shown in a bent or pivoted position that is not coplanar with the base portion 10. Conjugated polymer 30 is shown in an expanded state, thereby biasing articulating substrate 22 downward. FIGS. 4A and 4B also show the electrical contact layer 20 disposed between the articulating substrate 22 and the conjugated polymer 30. A portion of microelectrode 28 is also shown.

As noted above, the conjugated polymer 30 may be fabricated and conditioned such that it is coplanar with the base portion 10 in its first “steady state” position, i.e., without an electrical charge or voltage potential applied to conjugated polymer 30. As used in this application, an applied “electric charge” includes an applied voltage potential, an applied electric current, and/or an applied electric field. Alternatively, conjugated polymer 30 could be fabricated such that in its first steady state position it is pivoted away from substrate 10. Thus the expanded state of polymer 30, shown in FIG. 4A pivoting the articulating portion away from the base portion, could be its first steady state position. Depending on polymer conditioning and fabrication, however, FIG. 4A could also be depicting polymer 30 in its actuated second position, i.e., its position during application of an applied electric charge or voltage. Depending on the application, it may be advantageous, for example, for a device to be surgically attached or implanted with the articulating portion 12 actuated in a second position which is coplanar to the base, and then for articulating portion 12 to be extended or pivoted to a non-coplanar position in a first steady state position, after surgery is complete. In such an application, a voltage or electric charge would be applied to the conjugated polymer 30 during implantation and then removed in order to pivot the articulating portion 12 away from the base substrate. Of course in other applications, it may be preferable to have the articulating portions 12 biased to a coplanar position in a steady state and actuated to a non-coplanar pivoted position upon an applied electric charge or voltage potential. Further, it may be advantageous to actuate conjugated polymer 30 between different positions during insertion or implantation, i.e., pivoting articulating portion 12 between different positions as part of the medical procedure to optimize placement of the biological tissue interface, shown in FIG. 4A as microelectrode 28.

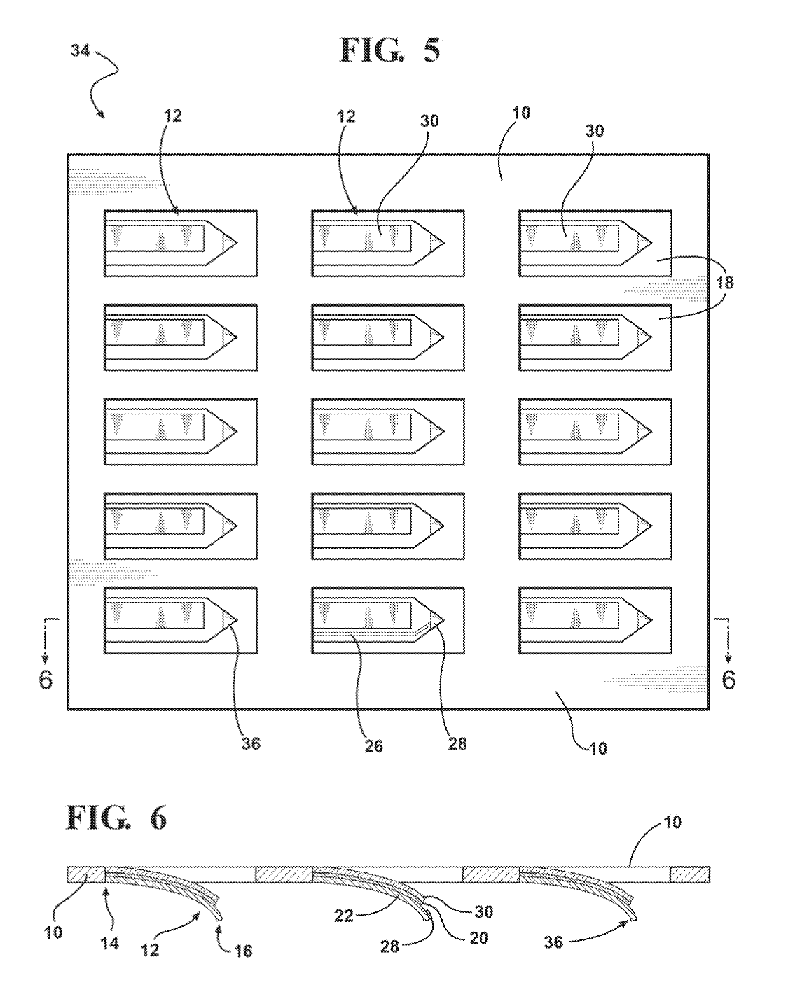

FIG. 5 illustrates a grid array 34 embodiment comprising a base portion 10, articulating portions 12, and open spaces 18. Articulating portions 12 are shown with conjugated polymer actuators 30 and biological tissue interfaces 36, which may or may not be neural interfaces. For simplicity, FIG. 5 omits electrical traces 24 to the polymer actuators 30 and all trace insulation layers. For illustration purposes, the bottom center articulating portion is shown with an electrical trace 26 connected to a microelectrode 28. The remaining distal ends of articulating portions 12 are shown with a generic biological tissue interface 36. It should be readily apparent, however, that grid array 34 may be fabricated with neural interfaces on the articulating portions, including neural interfaces comprising microelectrodes 28 or neural interfaces comprising another conducting surface, such as conjugated polymer 30 itself. Moreover, grid array 34 may have biological tissue interfaces that function primarily to secure grid array 34 to the surface of biological tissue, as more fully described below. In this 3×5 grid array, each of the articulating portions has a conjugated polymer actuator 30. In actual practice, and depending on the fabrication capabilities and the application desired, any number of articulating portions may be fabricated into a grid array 34, with various combinations and species of biological tissue interface sites. Further, the grid array could be formed in any pattern, not necessarily in rows and columns, and not necessarily with all of the articulating portions pointed in the same direction. Indeed, in some applications it may be preferable to have the articulating portions arranged opposably on the base portion, alternating in different directions, or fabricated in a circular arrangement with the articulating portions pivoted radially either away from the center of the grid or towards it. In another embodiment, grid array 34 could be attached to the bottom of a well for in vitro use. For example, grid array 34 could be adapted to cooperate with a microelectrode array within a well, available from various vendors including Multi Channel Systems, MCS GmbH, Aspenhaustraβe 21, 72770 Reutlingen, Germany, 07121 909250.

FIG. 6 is a sectional view of FIG. 5 showing grid array 34 with the articulating portions pivoted away from the base portion 10 in a non-coplanar position. FIG. 6 shows base portion 10, along with articulating portions 12, each having a proximal end 14 and distal end 16. The middle articulating portion 12 is shown with a microelectrode 28 at the distal end of articulating portion 12, as well as articulating substrate 22, electrical contact layer 20, and conjugated polymer 30 as the actuator. Again, at the distal end 16 of articulating portion 12, may be a microelectrode 28 or other conducting surface, or a biological tissue interface 36 that does not have a conducting surface. Biological tissue interface 36 could, for example, be configured to grasp tissue without the ability to interface neural tissue.

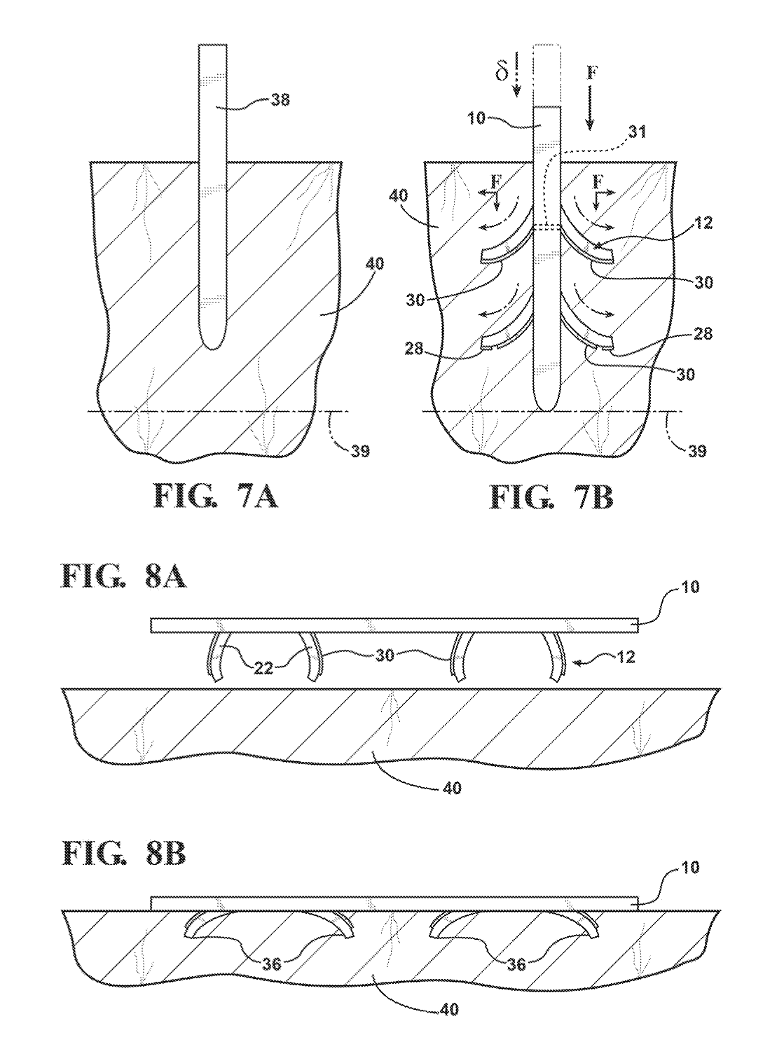

Turning to FIGS. 7A and 7B, a side view of a biological tissue interface device is shown implanted into biological tissue 40. FIG. 7A shows an implantable device 38 in a first position with articulating portions in coplanar position. FIG. 7B shows two pairs of articulating portions 12 pivoted away from base portion 10 within biological tissue 40 in an intra-tissue fashion. The pivoting action imparts a force F drawing implantable device 38 downward a distance δ. The pivoting action of articulating portion 12 also imposes a lateral (in this view) force upon the substrate of base portion 10. It may be desirable in some applications to have opposable pairs of articulating portions 12 arranged on the base portion 10 such that the lateral forces cancel each other out leaving only the downward force to draw the implantable device into the tissue to a desired insertion depth 39. The downward force may also be an applied external force inserting the implantable device 38 into tissue 40.

FIG. 7B also illustrates another embodiment whereby the polymer actuators 30 on articulating portions 12 can serve as a conductive path, or shunt, for neural signals. This may be desirable where neural tissue is damaged, interfering with the natural transmission of neural signals. As shown in FIG. 7B, the upper pair of articulating portions 12 have polymer actuators that extend left and right to the distal ends 16 of articulating portions 12. A conductive bridge 31 electrically connects both polymer actuators 30 in the pair. In their expanded state, the pivoted articulating portions 12 would extend their respective polymer actuators 30 away from base portion 10 into neural biological tissue 40 on the left side and right side of device 38, as viewed on FIG. 7B. Because the polymers are themselves conductive, the device thus positioned and in its steady state first position could serve as a neural shunt, allowing neural biological tissue 40 on the left side of device 38 to communicate with neural biological tissue on the right side of device 38. Thus device 38 configured as a polymer shunt or combination of shunts could restore damaged neural pathways.

The lower pair of articulating portions 12 in FIG. 7B are configured similarly to the articulating portions earlier described in FIGS. 3, 4 and 4A, where the microelectrodes 28 could be pivoted outward from base portion 12 and into biological tissue 40, to either stimulate or record neural tissue signals. Those skilled in the art will recognize many potential applications for such a configuration. For example, the left microelectrode 28 on the lower pair of articulating portions 12 could be used to receive a neural signal, the signal could then be directed to a system processor (not shown), e.g. for amplification, then the processed signal could be directed to the right microelectrode 28 to stimulate proximate neural tissue 40. It will also be apparent that implantable device 38 could be configured with any number of combinations of grasping, stimulating, recording, and neural shunt combinations.

FIG. 8A shows a side view of another embodiment of a biological tissue interface system. Articulating portions 12, comprising articulating substrates 22 and polymer actuators 30, extend downward from base portion 10, above biological tissue 40. FIG. 8B shows the interface system engaged with biological tissue 40 with the articulating portions 12 implanted within the biological tissue, in an intra-tissue fashion, and pivoted some distance back toward the base portion 10. Thus tissue interface portions 36 at the ends of the articulating portions 12 have grasped biological tissue and when pivoted back toward base portion 10 draw base portion 10 into an engagement position with biological tissue 40. Not shown in FIGS. 8A or 8B are neural interface portions, which are preferably articulating neural interfaces but are not necessarily articulating. Neural microelectrodes or other conducting surfaces could be fabricated on base portion 10. An embodiment such as in FIGS. 8A and 8B may be employed, for example, on the surface of the brain to function as ECOG electrodes.

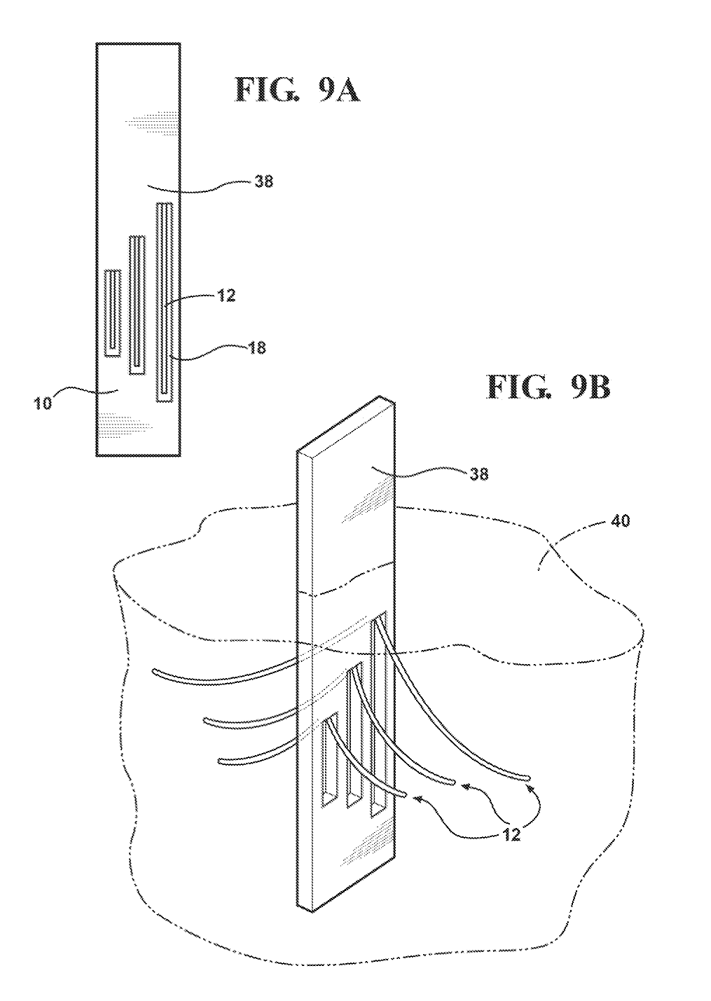

FIGS. 9A and 9B show another embodiment of an implantable device 38 with FIG. 9A showing a side view of base portion 10, articulating portions 12, and opening 18. FIG. 9B shows the implantable device 38 inserted into biological tissue 40 with articulating portions 12 pivoted away from base portion 10 in a non-coplanar state and engaging biological tissue 40 in an intra-tissue fashion. For simplicity, FIGS. 9A and 9B do not show details of the polymer actuator, electrical traces, microelectrodes, etc. This embodiment may be useful, for example, where engagement at different distances from the base portion site is desired, such as interfacing microelectrodes at the ends of articulating portions within a cortical layer at different distances. The architecture may also be useful if the implantable device 38 is to be used both for neural recording and stimulation. A similar architecture with varying distances from the base portion of the device may also be useful when employing the invention as a neural shunt as discussed above.

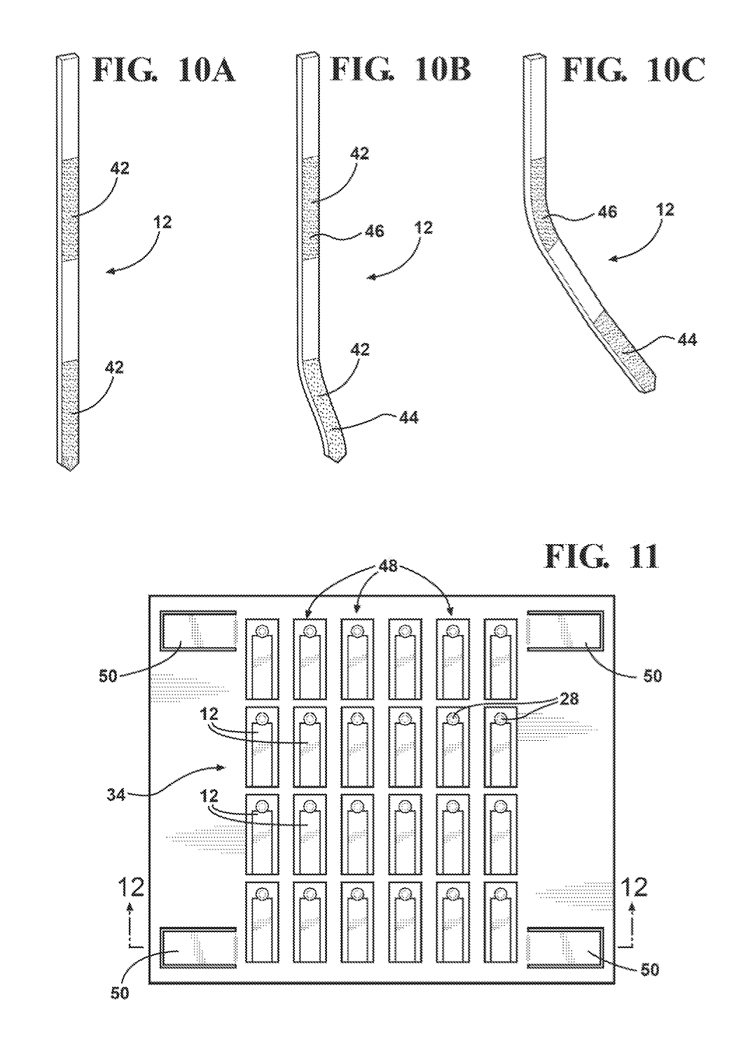

Depending on the dimensions of the articulating portion and the desired application, a plurality of conductive portions may be placed on a single articulating portion 12. FIG. 10 shows two conductive portions 42 on a single articulating portion 12. FIG. 10 shows a conjugated polymer actuator in the middle of the articulating portion in shank segment 46. Tip segment 44 may also be articulating by a polymer actuator. Depending on the application and limitations of the substrate end polymer, each articulating portion 12 could have two or more actuators 30 on it, and each such actuator could be actuated independently of other actuators on the same articulating portion. Further, one or more of the polymers themselves could be configured as conductive surfaces 42 for neural interfaces, or one or more microelectrodes with a substantially isolated electrical system for communication of neural signals could be fabricated on the shank segment 46 and/or tip segment 44. Further, such pluralities of actuators could be fabricated into a grid array.

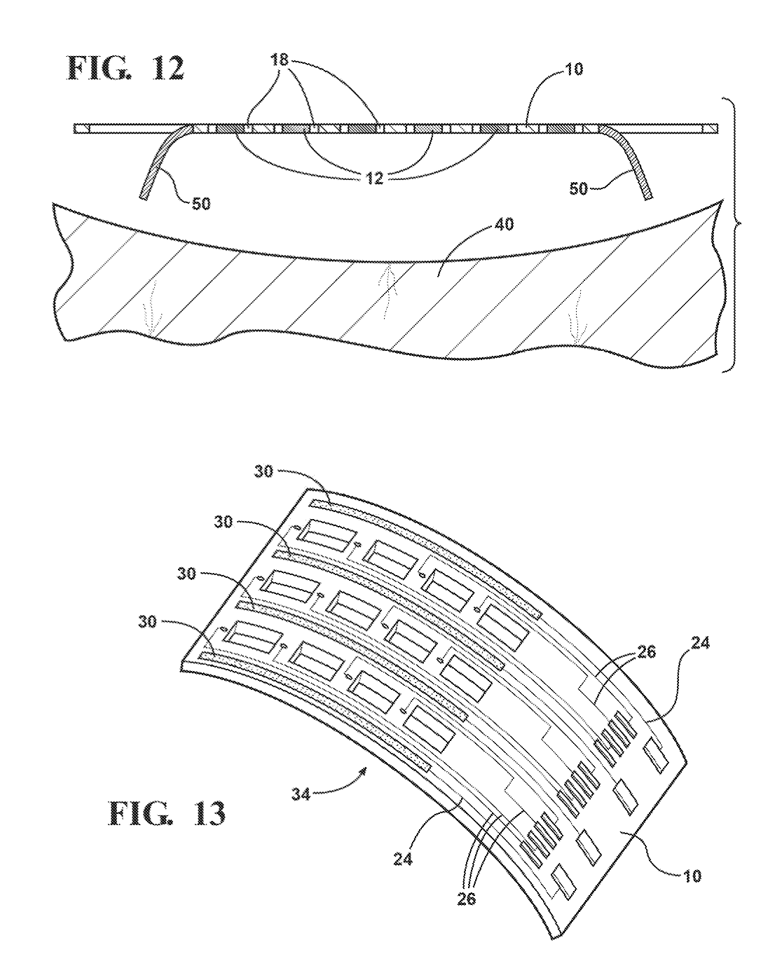

FIG. 11 shows another embodiment of a grid array 34, having multiple neural interfaces 48, articulating portions 12, and microelectrodes 28. FIG. 11 also shows four grasping interfaces 50. FIG. 12 is a sectional view of the same grid array 34 shown in FIG. 11, with grasping portions 50 shown (for illustration purposes) as pivoted away from base portion 10 in a non-coplanar position. For illustration purposes, articulating portions 12 are shown in FIG. 12 as not pivoted away from base portion 10. Biological tissue 40 might be, for example, the surface of the retina. Where the surface of neural tissue is curved, such as the retina, the distance between grasping portions 50 may be varied such that base portion 10 can be situated an optimal distance above the retina, for example, in order for neural interfaces 48 in a pivoted steady state first position to engage the retinal neural tissue at an optimal distance without damaging it. “Securing the base portion to biological tissue” as used in the claims includes engaging the exemplar grid array in FIG. 11 at an optimal distance to the retina neural tissue using intra-tissue grasping portions 50.

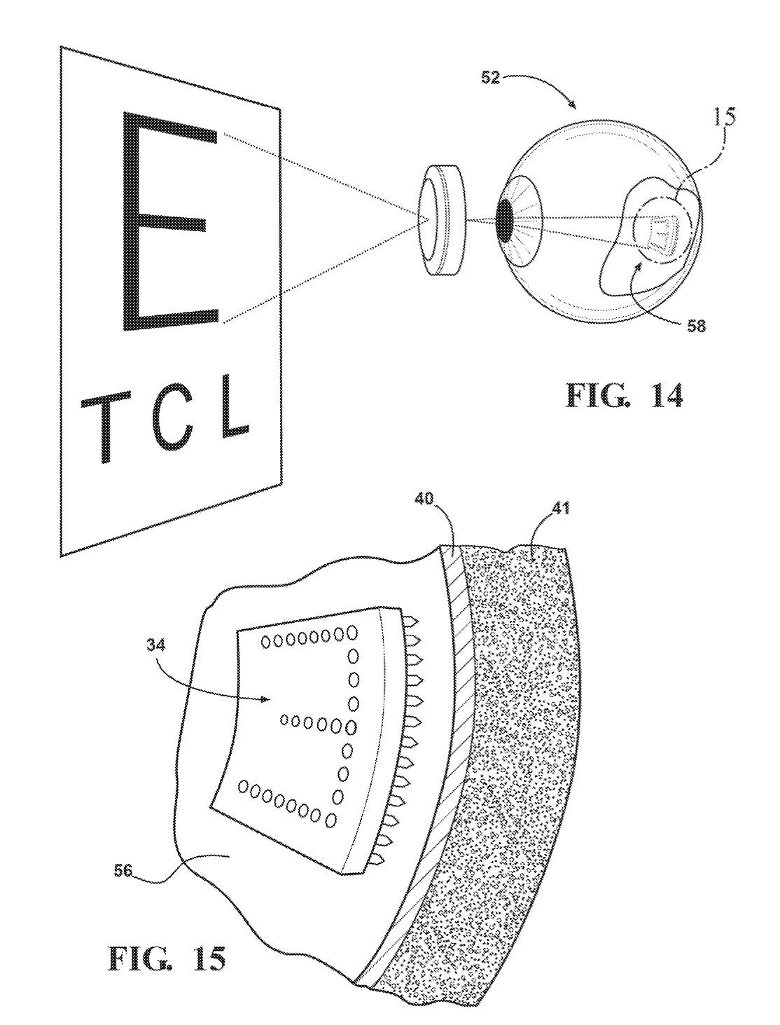

A retina application is shown in FIGS. 14 and 15, where video camera 54 replaces the pupil portion of the eye, communicating a signal representing a projected image 58 to the grid array 34 secured to the retinal wall surface 56. FIG. 15 shows an enlarged view of the retinal grid array 34 attached to the rear retinal wall surface 56, situated adjacent to biological tissue 40 and subretinal tissue 41.

FIG. 13 shows another embodiment of the invention in the form of a cuff electrode grid array that may be suitable for engaging peripheral nerves to an electrical system. Grid array 34 is configured such that base portion 10 has sufficient flexibility to be rolled up and around peripheral nerves via a plurality of conductive polymer actuators 30. Electrical traces 24 lead to the conjugated polymer actuators 30. Electrical traces 26 lead to microelectrodes that may be placed on base portion 10 as shown in FIG. 13, or attached to neural interfaces on articulating portions fabricated within grid array 34 (not shown). Those skilled in the art will appreciate that the neural interfaces may be placed directly on the base portion without a pivoting articulating portion, or that individual articulating portions could be fabricated into flexible grid array 34, similar to FIGS. 5, 6 and 11, such that the individual neural interfaces could be actuated to pivot away from base portion 10, while at the same time base portion 10 is rolled up via actuators 30 as shown in FIG. 13. Moreover, the roll-up grid array 34 could include a combination of microelectrodes on base portion 10 as well as on some or all articulating portions fabricated into flexible grid array 34. Moreover, the roll up grid array 34 could include neural interfaces where the conductive surface that engages with the neural tissue is the conjugated polymer 30 itself, effectively combining roles as an actuator and a neural interface.

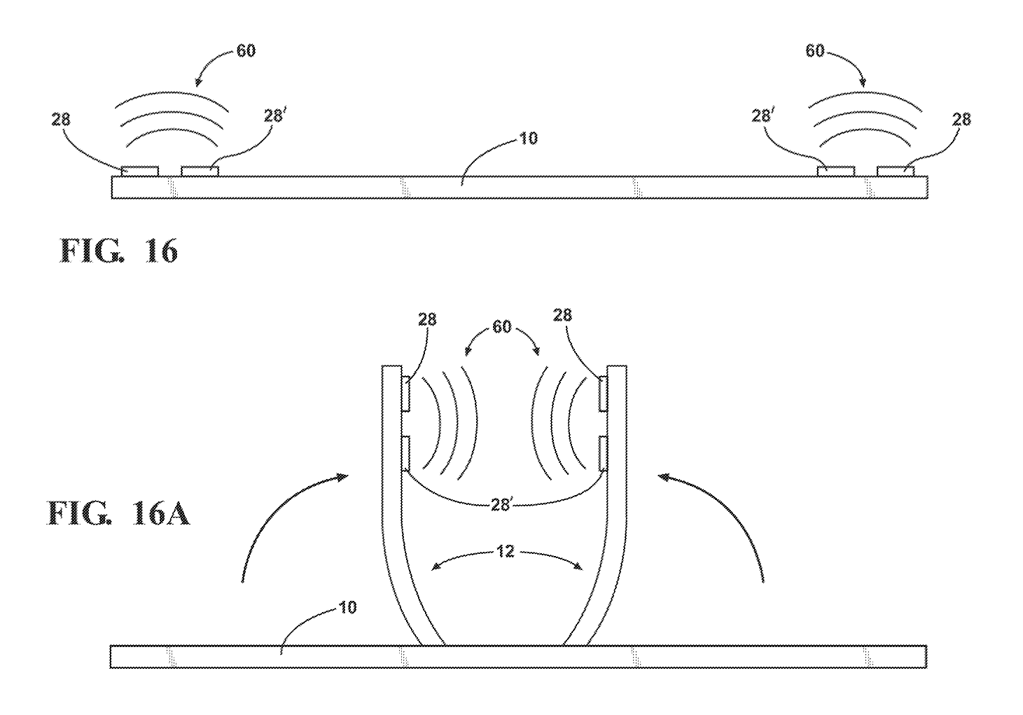

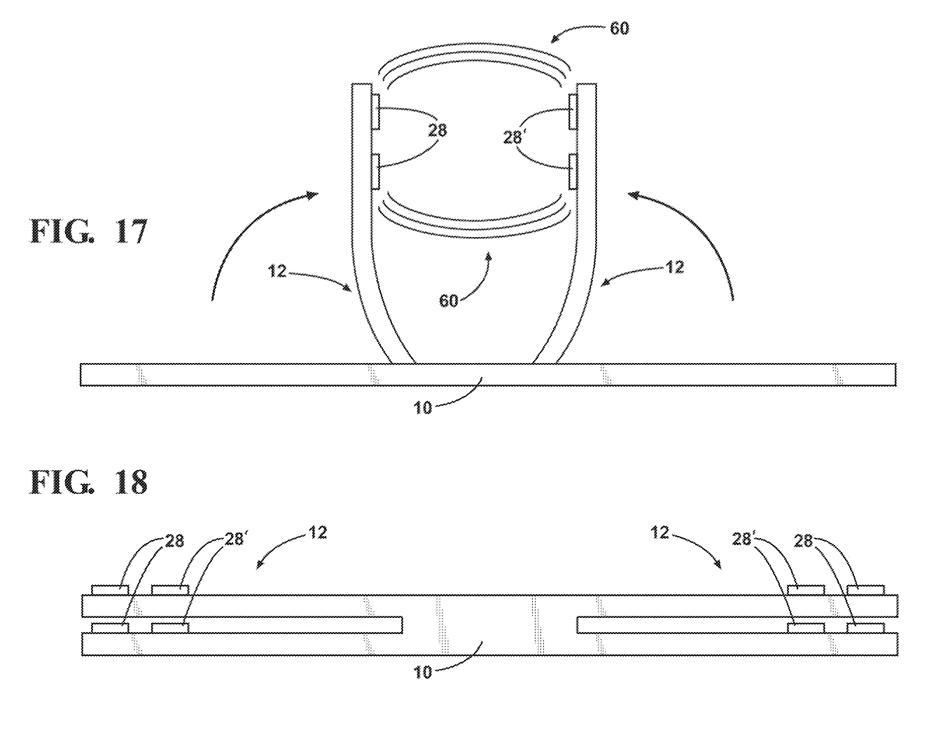

FIG. 16 shows another embodiment comprising a base portion 10 and two pairs of microelectrodes 28 and 28′ where 28 represents, for purposes of illustration, a positive electric charge or voltage potential and 28′ represents a negative electric charge or voltage potential. Stimulating current/voltage 60 could thus be applied to the pairs of microelectrodes 28 and 28′ in the planar position as shown in FIG. 16. The invention may be favorably employed by “steering” electric current to a position distant from base portion 10 by pivoting articulating portions 12 away from base portion 10, thereby steering the current to a distant position. As shown in FIG. 17, the neural interface system could be arranged such that a positive charge is applied to one or more microelectrodes 28 on a first articulating portion 12, and a negative charge is applied to one or more microelectrodes 28′ on a second articulating portion 12. The device could be fabricated to permit combinations of configuration current/voltage configurations 60 by, for example, modifying current or voltage polarity and/or magnitude to the electrical traces (not shown) connected to the microelectrodes.

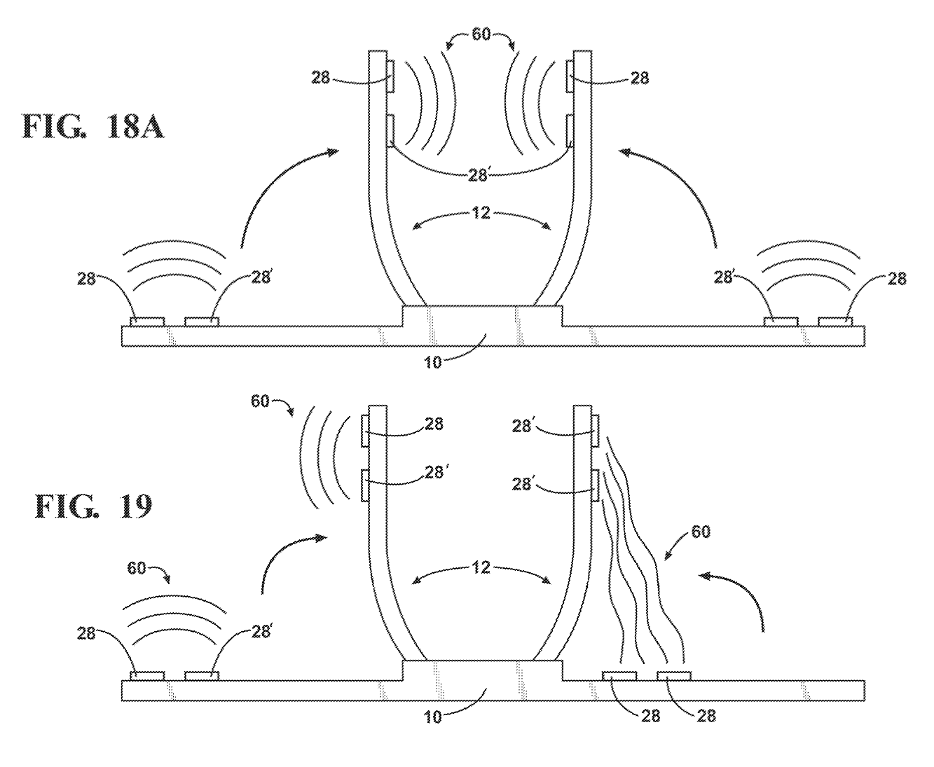

FIGS. 18 and 18A show another embodiment of stacked microelectrodes to enable “steerable” current/voltage 60. In this embodiment, microelectrodes 28 and 28′ on articulating portions 12 may be pivoted away from base portion 10, while other microelectrodes remain active on base portion 10. It will be apparent that additional articulating portions 12 of differing lengths could pivot at different points from the base portion 10, and in different directions, each configuration having a wide variety of polarity, electrical magnitude, and orientation combinations. FIG. 19 shows two such alternative arrangements.

The disclosed device and method can actuate an arm (the articulating portion) carrying a conductive surface such as a microfabricated electrode (either a sensor or transmitter) that can be manipulated and moved in vivo on the biological tissue/nerve, either implanted into the tissue or at the tissue surface, to maintain or optimize contact. Applications may include enhancing grid electrodes such as ECOG electrodes, retinal implants, peripheral neural applications, and cuff electrodes. The common desire in such applications is to make contact between a microelectrode site and the targeted nerve/tissue. By adding the ability to adjust the contact distance and pressure, the desired application may be enhanced by improving the signal to noise ratio and decreasing the amplitude for stimulation therapy, thereby reducing deleterious effects.

Some researchers are investigating neurons mechanically twitching as a result of stimulus. Similarly, conjugated polymers are known for their impedance changes due to strain. The articulating portions could thus also function as a pressure sensor. A pressure on the articulating portion could translate to a stress on the conjugated polymer to which it is coupled, causing a change in its impedance. Such a device could thus be adapted to detect tissue motion and fluctuation both at the microlevel or grossly.

The MEMS device may be microfabricated. For example, specific regions of a microfabricated base substrate may be embedded or coated with a conjugated polymer. This can be done in a variety of ways. One method is to mechanically fabricate the base substrate and the articulating portion substrate, then apply a conjugated polymer to a portion of the articulating portion substrate (i.e. by physical vapor deposition, electrochemical deposition, or chemical deposition) thereby creating a bilayer articulating portion. A number of materials may be used for the base portion and articulating portion substrates. Traditional MEMS materials such as silicon and/or flexible polymers may be used for the base portion substrate. For the articulating portion substrate, the same materials may be used, preferably having more elasticity than the base portion substrate. In addition, resorbable substrate materials such as polyethylene glycol may be used, in whole or in part, on either substrate portion. Microfabrication techniques are known to those skilled in the art. By way of example only, a device might be formed using two different thicknesses of parylene for the base and articulating portion substrates. In such example, the parylene substrate may be coated with a thin layer of metal, such as Cr/Au on the order of hundreds of angstroms) for polymerization. Continuing with the same example, a layer of PPy(DBS) may then be galvanostatically polymerized on the Au layer of the articulating substrate with a deposition current density preferably of 1 mA/cm2 or less in a solution of 0.1 M pyrrole and 0.1 M DBS using a galvano/potentiostat. An Ag—Ag/Cl reference electrode and porous carbon counter electrode may be used. Further information on MEMS microfabrication methods and techniques may be found at Smela, E., “Microfabrication of PPy microactuators and other conjugated polymer devices,” Journal of Micromechanics and Microengineering, vol. 9, no. 1, pp. 1-18 (1999), which is incorporated by reference in its entirety.

Conjugated polymer actuators cause substrate deflection from the mechanical strain induced from the polymer swelling and shrinking. Variables determining the features that can be actuated successfully include substrate material properties, substrate dimensions, and actuator strain and dimensions. Variables affecting actuation performance include bilayer actuation force, resulting in deflection, and substrate buckling due to insertion forces, and biological environment, including ambient ionic concentrations. Therefore, the combination of the substrate bending stiffness and insertion and buckling forces should be considered while designing actuators tailored for the specific applications. When provided with substrate dimensions, actuators for electrode projections can be designed to fulfill a variety of application specifications. Conjugated polymer actuation strain is subject to several variables including polymer and dopant combinations, polymerization settings, actuating environment, redox speed, and actuator dimensions. Although the strain from these actuators are small in comparison with traditional actuation modalities, the typical deflections needed in neural interfacing applications is in the millimeter and micron scale and the power required to actuate the disclosed devices are favorable over other actuation modalities.

The specifications for electrode projections can be determined by the desired spread from the base substrate. Projections may be made from various substrate materials and can design their dimensions to satisfy targeted specifications. Application or fabrication considerations that impose limitations on the substrate material or particular dimensions can still vary the remaining independent variables including actuator thickness to produce more force to achieve their desired deflections.

This work introduces,the feasibility and mechanical considerations of conjugated polymers for use in a variety of neural interfacing applications. Electrode sites with individual projections can be actuated from the surface of their underlying substrate to reduce their distance to their interfacing target. This feature may improve the neural interface by optimizing the electrode-tissue distance or by distancing the electrode site from the larger substrate when favored. In another embodiment, the invention may be favorably employed as a neural shunt. In another embodiment, the invention may be favorably employed as a biological tissue grasping device. In another embodiment, the invention may be favorably employed to steer current/voltage to neural tissue. In another embodiment, the invention may be favorably employed in the bottom of a microelectrode well for in vitro use to engage with biological tissue and/or cells.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention; all such modifications are intended to be included within the scope of the invention.

What is claimed is:

1. A neural tissue interface system comprising:
a base portion;
an articulating portion having a proximal end attached to the base portion and a free distal end;
an actuator operably coupled to the articulating portion, said actuator comprising a polymer that in response to an applied electric charge applies a force to pivot the articulating portion, relative to the base portion, from a first position to a second position; and
a neural interface portion located proximate the distal end of the articulating portion and comprising a conductive surface engageable with neural tissue.

2. The neural tissue interface system of claim 1, wherein the conductive surface comprises the polymer.

3. The neural tissue interface system of claim 2, further comprising at least one pair of articulating portions opposably arranged on the base portion and actuated by polymer actuators, with the pair of said polymer actuators conductively connected by an electrical trace.

4. The neural tissue interface system of claim 1, wherein the conductive surface comprises a microelectrode that is substantially electrically isolated from the polymer.

5. The neural tissue interface system of claim 1, further comprising a plurality of microelectrodes on the articulating portion.

6. The neural tissue interface system of claim 5, wherein an electric charge may be applied between at least two of said microelectrodes on the articulating portion.

7. The neural tissue interface system of claim 1, further comprising a conductive surface on the base portion, wherein an electric charge may be applied between the conductive surface on the base portion and the conductive surface on the neural interface portion.

8. The neural tissue interface system of claim 1, wherein said articulating portion comprises a plurality of actuators that may each be actuated independently.

9. The neural tissue interface system of claim 1, wherein the system comprises a plurality of actuators operably coupled to a plurality of articulating portions, and a plurality of neural interface portions.

10. The neural tissue interface system of claim 9, wherein at least two of the plurality of actuators may be actuated independently.

11. The neural tissue interface system of claim 9, wherein the conducting surfaces on at least two of the neural interface portions may be controlled independently.

12. The neural tissue interface system of claim 9, wherein an electric charge may be applied via the conducting surfaces between at least two different articulating portions.

13. The neural tissue interface system of claim 1, wherein the articulating portion in said first position is substantially coplanar with the base portion.

14. The neural tissue interface system of claim 1, wherein the articulating portion in said second position is substantially coplanar with the base portion.

15. The neural tissue interface system of claim 1, wherein the base portion comprises a first electrical trace connected to the polymer on the actuator.

16. The neural tissue interface system of claim 15, wherein the base portion comprises a second electrical trace substantially electrically isolated from the polymer and connected to a microelectrode on the interface portion.

17. A neural tissue interface system, comprising:
a base portion comprising a substrate, a first electrical trace connected to a electrical charge source, and a second electrical trace to communicate neural signals;
an articulating portion having a proximal end attached to the base portion and a free distal end;
an actuator operably coupled to the articulating portion, said actuator comprising a polymer that is connected to the first electrical trace and that in response to an applied electric charge applies a force to pivot the articulating portion, relative to the base portion, from a first position to a second position, and that in the absence of said applied electric charge biases the actuator toward the first position; and
a neural interface portion situated proximate the distal end of the articulating portion and in communication with neural tissue and with the second electrical trace.

18. The neural tissue interface system of claim 17, further comprising an intra-tissue articulating portion operably coupled with a polymer actuator that, in response to a change in electric charge, secures the neural interface portion to the neural tissue by grasping biological tissue into which the articulating portion has been implanted.

19. A biological tissue interface system, comprising:
a base portion;
an intra-tissue articulating portion on said base portion; and
an actuator operably coupled to said articulating portion, said actuator comprising a polymer that in response to an applied electric charge applies a force to pivot the articulating portion, relative to the base portion, to grasp biological tissue into which the articulating portion has been implanted thereby securing the base portion to the biological tissue.

20. The biological tissue interface system of claim 19, further comprising a neural interface that is substantially electrically isolated from the polymer.

21. A method of communicating between neural tissue and an electrical system, comprising the steps of:
providing a system base portion comprising a substrate, a first electrical trace connected to a electric charge source, and a second electrical trace to communicate neural signals;
providing a system articulating portion having a proximal end attached to the base portion and a free distal end;
providing a system actuator operably coupled to the articulating portion, said actuator comprising a polymer that is connected to the first electrical trace;
providing a neural interface portion comprising a conductive surface situated proximate the distal end of the articulating portion and connected to the second electrical trace;
applying an electric charge to the polymer via the first electrical trace;
pivoting the articulating portion, relative to the base portion, from a first position to a second position;
placing the distal end of the articulating portion near targeted neural tissue;
removing the applied electric charge;
biasing the actuator and pivoting the articulating portion to move the neural interface portion toward said first position and closer to the targeted neural tissue;
communicating a signal between the targeted neural tissue and the system via the conductive surface and second electrical trace.
